(19) United States
(12) Patent Application Publication
(10) Pub. No.: US 2007/0048764 A1
(43) Pub. Date: Mar. 1, 2007

(54) Indicator polynucleotide controls

(76) Inventors: Jonathan Bingham, San Francisco, CA (US); Subha Srinivasan, Greenbrae, CA (US)

Correspondence Address: PERKINS COIE LLP, P.O. BOX 2168, MENLO PARK, CA, 94026, US

(21) Appl. No.: 11/445,867

(22) Filed: Jun. 2, 2006

Related U.S. Application Data
(60) Provisional application No. 60/710,559, filed on Aug. 23, 2005.

(51) Int. Cl. C12Q 1/68; G06F 19/00; C12M 1/34

(52) U.S. Cl. 435/6; 435/287.2; 702/20

(57) ABSTRACT

Methods of selecting indicator polynucleotide controls for use, for example, in determining amounts of indicated polynucleotides in a sample are described. Devices that include indicator polynucleotide controls and data representations relevant to them are also described. A storage medium comprising data obtained from a study using a nucleotide array that includes one or more of the indicator polynucleotide controls is also described.

### CROSS-REFERENCE TO RELATED APPLICATIONS

This application claims the benefit of U.S. Provisional Application No. 60/710,559, filed Aug. 23, 2005, incorporated herein by reference in its entirety.

### TECHNICAL FIELD

The subject matter described herein relates to nucleotide arrays for detection of nucleic acids in a sample and to indicator polynucleotides for use in nucleotide arrays.

### BACKGROUND

An indicator polynucleotide, such as an oligonucleotide, may be used to determine an amount of a target polynucleotide in a sample by attaching the indicator polynucleotide, or probe, to solid surface such as a nucleotide array, placing two or more indicator polynucleotides in a well on a multi-well plate for use in a polymerase chain reaction, or via another method. The measured amount of the target polynucleotide may include some undesired amount of another polynucleotide caused by a variety of factors, such as spurious binding to homologues and other polynucleotides. It would be useful to measure the undesired amount of a non-target polynucleotide in order to differentiate it from the amount of a target polynucleotide. This information could be used, for example, to compute a signal to noise ratio, to subtract noise, to identify an indicated polynucleotide expressed above the level of background reading or noise, as well as for other purposes.

An indicator polynucleotide may indicate the presence of an exon-exon junction polynucleotide in a sample. Such an indicator polynucleotide may be used, for example, to measure the amount of an exon-exon junction polynucleotide of an alternately spliced gene in a sample. When using an exon-exon junction indicator polynucleotide, some undesired signal may result from binding of the indicator polynucleotide to other polynucleotides in the sample, including “half-binders”—polynucleotides containing one or the other, but not both, of the exon polynucleotides at the exon-exon junction.

By way of illustration, consider the following example. An exon-exon junction indicator polynucleotide (SEQ ID NO:1) comprised of an indicator for exon A (SEQ ID NO:2) and an indicator for exon B (SEQ ID NO:3):

(SEQ ID NO:1)

AGAAGTATGGCCCGATTTAC AGATGGACTTGCCACCAGTG

(SEQ ID NO:2)

Exon A Indicator

(SEQ ID NQ:3)

Exon B Indicator

SEQ ID NO:1 is capable of hybridizing to a target polynucleotide, for detection of an indicated polynucleotide in a sample, the indicated polynucleotide having the sequence identified as SEQ ID NO:1. The indicated polynucleotide is comprised of an exon A polynucleotide (SEQ ID NO:2) and an exon B polynucleotide (SEQ ID NO:3).

The sample may also contain a “half-binder” polynucleotide AC (SEQ ID NO:4) comprised of an exon A polynucleotide (SEQ ID NO:2) and an exon C polynucleotide (SEQ ID NO:5):

(SEQ ID NO:4)

AGAAGTATGGCCCGATTTAC AGGGAGAAGCTCGGCAAC

(SEQ ID NO:2)

Exon A Polynucleotide

(SEQ ID NO:5)

Exon C Polynucleotide

The sample may also contain a “half-binder” polynucleotide DB (SEQ ID NO:6) comprised of an exon D polynucleotide (SEQ ID NO:7) and an exon B polynucleotide (SEQ ID NO:3):

(SEQ ID NO:6)

AGTAGCATAGCTCGATTTAC AGATGGACTTGCCACCAGTG

(SEQ ID NO:7)

Exon D Polynucleotide

(SEQ ID NO:3)

Exon B Polynucleotide

The presence of “half-binder” polynucleotide AC (SEQ ID NO:4) in the sample may interfere with the measured amount of the indicated polynucleotide (SEQ ID NO:1) since exon A indicator (SEQ ID NO:2) will bind to a target sequence that indicates exon A polynucleotide (SEQ ID NO:2), even though exon C polynucleotide (SEQ ID NO:5) is not indicated by exon B indicator (SEQ ID NO:3). It would be desirable to measure this amount in order to differentiate it from the measured amount of the indicated polynucleotide. Similarly, the presence of “half-binder” polynucleotide DB (SEQ ID NO:6) may interfere with the measured amount of the indicated polynucleotide since exon B indicator (SEQ ID NO:3) will bind to a target sequence that indicates exon B polynucleotide, even though exon D polynucleotide (SEQ ID NO:7) is not indicated by exon A indicator (SEQ ID NO:2). It would be desirable to measure this amount in order to differentiate it from the amount measured for the indicated polynucleotide.

An indicator polynucleotide may indicate the presence of an exon-intron junction polynucleotide in a sample. Such an indicator polynucleotide may be used, for example, to measure the amount of exon-intron junction polynucleotides from pre-mRNA in a sample. When using an exon-intron junction indicator polynucleotide, some undesired signal may result from binding of the indicator polynucleotide to other polynucleotides in the sample, including polynucleotides containing the indicated exon polynucleotide but not the indicated intron polynucleotide. By way of another example, consider an exon-intron junction indicator polynucleotide (SEQ ID NO:8):

| | |
|---|---|
| AGGTCGGAGTCAACGG GTAACATTGACTGTCAA | (SEQ ID NO:8) |
| Exon A Indicator | (SEQ ID NO:9) |
| Intron Indicator | (SEQ ID NO:10) |

SEQ ID NO:8 is capable of hybridizing to a target polynucleotide, for detection of an indicated polynucleotide in a sample, the indicated polynucleotide having the sequence identified as SEQ ID NO:8. The indicated polynucleotide is comprised of an exon A polynucleotide (SEQ ID NO:9) and an exon B polynucleotide (SEQ ID NO:10).

The sample may also include a “half-binder” polynucleotide AB:

(SEQ ID NO:11)

AGGTCGGAGTCAACGG AGATGGACTTGCCACCAGTG

(SEQ ID NO:9)

Exon A Polynucleotide

(SEQ ID NO:3)

Exon B Polynucleotide

The presence of “half-binder” polynucleotide AB (SEQ ID NO:11) may interfere with the measured amount of the indicated polynucleotide since exon A indicator will bind to a target sequence that indicates the presence of exon A polynucleotide, even though exon B polynucleotide (SEQ ID NO:3) is not indicated by intron indicator (SEQ ID NO:10). It would be desirable to measure this amount in order to differentiate it from the amount of the desired indicated polynucleotide.

An indicator polynucleotide may indicate the presence of an intron-exon junction polynucleotide in a sample. Such an indicator polynucleotide may be used, for example, to measure the amount of intron-exon junction polynucleotides from pre-mRNA in a sample. When using an intron-exon junction indicator polynucleotide, some undesired signal may result from binding of the indicator polynucleotide to other polynucleotides in the sample, including polynucleotides containing the indicated exon polynucleotide but not the indicated intron polynucleotide.

For example, consider an intron-exon junction indicator polynucleotide having the sequence:

| | |
|---|---|
| ACATACGTTCTATTTAG ATTTGGTCGTATTGGGCG | (SEQ ID NO:12) |
| Intron Indicator | (SEQ ID NO:13) |
| Exon A Indicator | (SEQ ID NO:14) |

SEQ ID NO:12 is capable of hybridizing to a target polynucleotide, for detection of an indicated polynucleotide in a sample, the indicated polynucleotide having the sequence identified as SEQ ID NO:12. The indicated polynucleotide is comprised of an intron polynucleotide (SEQ ID NO:13) and an exon A polynucleotide (SEQ ID NO:14).

The sample also may contain a “half-binder” polynucleotide BA:

(SEQ ID NO:15)

AGAAGTATGGCCCGATTTAC ATTTGGTCGTATTGGGCG

(SEQ ID NO:2)

Exon B Polynucleotide

(SEQ ID NO:14)

Exon A Polynucleotide

The presence of “half-binder” polynucleotide BA (SEQ ID NO:15) may interfere with the measured amount of the indicated polynucleotide, since exon A indicator binds a target that indicates the presence of exon A polynucleotide, and therefore the presence of the intron-exon junction indicated polynucleotide, even though exon B polynucleotide (SEQ ID NO:2) is not indicated by intron indicator (SEQ ID NO:13). It would be desirable to measure this amount in order to differentiate it from the measured amount of the indicated polynucleotide.

An indicator polynucleotide may indicate the presence of an exon polynucleotide in a sample. Some undesired signal may result from binding of the indicator polynucleotide to other polynucleotides in the sample. It would be desirable to measure this amount in order to differentiate it from the desired amount from the indicated exon polynucleotide.

It would be desirable to represent and analyze the data resulting from indicator polynucleotides, indicator polynucleotide controls, and their interactions, in order to more accurately measure the presence of indicated polynucleotides in samples and to more accurately draw biological inferences.

The foregoing examples are intended to be illustrative and not exclusive. Other features of the subject matter will become apparent to those of skill in the art upon a reading of the specification and a study of the sequences.

### BRIEF SUMMARY

In one aspect, a nucleotide array is provided. The array is comprised of (i) an indicator polynucleotide for a target polynucleotide in a sample, the indicator polynucleotide selected from the group consisting of an indicator polynucleotide for an exon-exon junction, an indicator polynucleotide for an exon-intron junction, an indicator polynucleotide for an intron-exon junction, indicator polynucleotide for an exon and an indicator polynucleotide for an intron, and (ii) an indicator polynucleotide control for the indicator polynucleotide, the indicator polynucleotide control selected from the group consisting of a swap control and a balanced mutation control.

In another aspect, a nucleotide array comprising a plurality of pairs of indicator polynucleotides is provided, each pair comprising: (i) an indicator polynucleotide for a target polynucleotide in a sample, the indicator polynucleotide selected from the group consisting of an indicator polynucleotide for an exon-exon junction, an indicator polynucleotide for an exon-intron junction, an indicator polynucleotide for an intron-exon junction, indicator polynucleotide for an exon and an indicator polynucleotide for an intron, and (ii) an indicator polynucleotide control for the indicator polynucleotide, the indicator polynucleotide control selected from the group consisting of a swap control and a balanced mutation control.

In one embodiment, the nucleotide array comprises at least about 100 of such pairs.

In another aspect, a storage medium is provided, where the storage medium comprises data obtained from a study using a nucleotide array as described above.

In one embodiment, the data is stored in a portion of a computer file. In another embodiment, the data is stored in one or more computer files.

In yet another aspect, a method of determining an expression level for a target exon-exon junction, exon-intron junction, or intron-exon junction polynucleotide in a sample is provided. The method comprises obtaining expression level data for a sample for (i) an indicator polynucleotide for a target polynucleotide in the sample, the indicator polynucleotide selected from the group consisting of an indicator polynucleotide for an exon-exon junction, an indicator polynucleotide for an exon-intron junction, an indicator polynucleotide for an intron-exon junction, an indicator polynucleotide for an exon, and an indicator polynucleotide for an intron, and (ii) an indicator polynucleotide control for the indicator polynucleotide, the indicator polynucleotide control selected from the group consisting of a swap control and a mutation control. Then, a mathematical algorithm is applied to the expression level data to determine an expression level for the target polynucleotide in the sample.

In still another embodiment, a method of determining if a splice variant of a gene is expressed in a sample is provided. The method comprises obtaining expression level data for a sample for (i) an indicator polynucleotide for a target polynucleotide in the sample, the indicator polynucleotide selected from the group consisting of an indicator polynucleotide for an exon-exon junction, an indicator polynucleotide for an exon-intron junction, an indicator polynucleotide for an intron-exon junction, an indicator polynucleotide for an exon, and an indicator polynucleotide for an intron, and (ii) an indicator polynucleotide control for the indicator polynucleotide, the indicator polynucleotide control selected from the group consisting of a swap control and a mutation control. Then, a mathematical algorithm is applied to the expression level data to determine if the splice variant of the gene is expressed in the sample.

In another aspect, a method of determining an expression level for one or more expected splice variants of a gene in a sample is provided. The method comprises obtaining expression level data for a sample for (i) an indicator polynucleotide for a target polynucleotide in the sample, the indicator polynucleotide selected from the group consisting of an indicator polynucleotide for an exon-exon junction, an indicator polynucleotide for an exon-intron junction, an indicator polynucleotide for an intron-exon junction, an indicator polynucleotide for an exon, and an indicator polynucleotide for an intron, and (ii) an indicator polynucleotide control for the indicator polynucleotide, the indicator polynucleotide control selected from the group consisting of a swap control and a mutation control. Then, a mathematical algorithm is applied to the expression level data to determine an expression level for each of the expected splice variants of the gene in the sample.

### DETAILED DESCRIPTION

Before the present subject matter is described, it is to be understood that it is not limited to particular embodiments described, as such may, of course, vary. It is also to be understood that the terminology used herein is for the purpose of describing particular embodiments only, and is not intended to be limiting, since the scope of the disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. Although any methods and materials similar or equivalent to those described herein can be used in the practice or testing of the described subject matter, some potential and preferred methods and materials are described herein.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an indicator polynucleotide” includes a plurality of such indicator polynucleotides.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Accordingly, and in one embodiment, indicator polynucleotide controls are described, where the indicator polynucleotide controls can be used as controls for another indicator polynucleotide. A plurality of such controls for a plurality of indicator polynucleotides can be used, as will be described. Selection of the controls can be done by a human or a computer software program using the indicator polynucleotide control (IPC) method described herein, or the method for selecting indicator polynucleotide controls may be used in hardware, such as a polynucleotide synthesizer.

A device designed based on the IPC method may comprise one or more polynucleotide pairs having an indicator polynucleotide and an indicator polynucleotide control immobilized on a solid surface, such as an oligonucleotide array. The device may comprise 100 or more such pairs, or 1000 or more pairs, or 10,000 or more pairs, or 1,000,000 or more pairs. Another device designed and constructed using the IPC method may comprise one or more polynucleotide pairs consisting of an indicator polynucleotide and an indicator polynucleotide control for use as primers in polymerase chain reactions (PCR). The primers may be placed in wells in a multi-well plate, such as a 96- or 384-well plate, or another container, substrate, or device for arranging primers in known locations.

The IPC method is illustrated by way of exemplary embodiments, where an indicator polynucleotide control for use in conjunction with an indicator polynucleotide for an indicated polynucleotide having, for example, an exon-exon junction, an exon-intron junction, an intron-exon junction, an exon, or an intron, is designed. Collectively, and more generally, the IPC method is suited for detection of an indicated polynucleotide that comprises at least a junction selected from an exon-exon junction, and exon-intron junction, and an intron-exon junction. The method is additionally suited for use in detecting indicated polynucleotides that do not have such an junction, for example polynucleotides with one or more exons or introns.

Exon-Exon Junction Indicator Polynucleotide Controls

In the case of an exon-exon junction indicator polynucleotide, the IPC method, in one embodiment, provides an indicator polynucleotide control having a sequence comprised of two portions, a first portion that indicates a first exon in an exon-exon junction and a second portion that indicates a second exon in the exon-exon junction. For example:

| | |
|---|---|
| (1) | Exon-exon junction indicator polynucleotide: |

| | |
|---|---|
| AGGTCGGAGTCAACGG ATTTGGTCGTATTG | (SEQ ID NO:16) |
| GGCG | |
| Exon A Indicator | (SEQ ID NO:9) |
| Exon B Indicator | (SEQ ID NO:14) |

| | |
|---|---|
| (1a) | Exon-exon junction indicator polynucleotide |
| | control: |

| | |
|---|---|
| ATTTGGTCGTATTGGGCG AGGTCGGAGTCA | (SEQ ID NO:17) |
| ACGG | |
| Exon B Indicator | (SEQ ID NO:14) |
| Exon A Indicator | (SEQ ID NO:9) |

In the exon-exon junction indicator polynucleotide control (SEQ ID NO:17), the polynucleotide indicating the first exon, Exon A Indicator (SEQ ID NO:9), is positioned after the polynucleotide indicating the second exon, Exon B Indicator (SEQ ID NO:14), relative to the original positions of these exons in the exon-exon junction indicator polynucleotide (SEQ ID NO:16). Exon A Indicator (SEQ ID NO:9) has a 5′ end and a 3′ end, and Exon B Indicator (SEQ ID NO:14) has a 5′ end and a 3′ end. The 5′ end of Exon A Indicator (SEQ ID NO:9) is adjacent the 3′ end of Exon B Indicator (SEQ ID NO:14) in the indicator polynucleotide control (SEQ ID NO:17). In contrast, in the original exon-exon junction indicator polynucleotide (SEQ ID NO:16), the 5′ end of Exon B Indicator (SEQ ID NO:14) is adjacent the 3′ end of Exon A Indicator (SEQ ID NO:9). In essence, the position of the portions of the exon-exon junction indicator polynucleotide control that identify the exons of the indicator polynucleotide are exchanged or “swapped”, relative to the position of the portions in the exon-exon junction indicator polynucleotide. An exon-exon junction indicator polynucleotide control as exemplified by the hypothetical SEQ ID NO:17 is referred to herein as a “swap control.”

The relative lengths of Exon A Indicator and Exon B Indicator may be unequal, with the length of Exon A Indicator less than the length of Exon B Indicator, or the length of Exon A Indicator greater than the length of Exon B Indicator. It will also be appreciated that the length of Exon A Indicator can be equal to the length of Exon B Indicator. U.S. Publication No. 2004/0076959, incorporated by reference herein, describes a method for adjusting the relative lengths of portions of an indicator polynucleotide to optimize binding of an exon-exon junction or an exon-intron junction indicator polynucleotide to a target.

The indicator polynucleotide control in this embodiment is also referred to herein as a ‘swap control’ for the exon-exon junction indicator polynucleotide, in the sense that the portions of the indicator polynucleotide control that identify the exons of the exon-exon junction indicator polynucleotide are exchanged, relative to their positions in the exon-exon junction indicator polynucleotide. The exon-exon junction indicator polynucleotide comprises a first portion that indicates the 3′ end of a first exon and a second portion that indicates the 5′ end of a second exon. The first portion precedes the second portion. The swap control comprises these same two portions, but ‘swapped’, so that the second portion comes before the first portion.

The exon-exon junction indicator polynucleotide may contain one or more additional polynucleotides that do not indicate either exon in the exon-exon junction. For example, the indicator polynucleotide may be formed by pre-pending a polynucleotide PA before Exon A Indicator, inserting a polynucleotide PAB between Exon A Indicator and Exon B Indicator, or appending a polynucleotide PB after Exon B Indicator. In one embodiment, the IPC method creates an exon-exon junction indicator polynucleotide control that shuffles the order of the constituent polynucleotides, PA, Exon A Indicator, PAB, Exon B Indicator, and PB. In one embodiment, the indicator polynucleotide control includes all of the constituent polynucleotides. In another embodiment, the indicator polynucleotide control includes Exon A Indicator and Exon B Indicator but does not include all of the other constituent polynucleotides. In another embodiment, the IPC method inserts one or more nucleotides at the exon-exon junction. In another embodiment, the IPC method deletes one or more nucleotides at the exon-exon junction.

In another embodiment the IPC method comprises providing an indicator polynucleotide control that includes reverse polynucleotides, reverse complement polynucleotides, or complement polynucleotides for Exon A Indicator, Exon B Indicator, or other polynucleotides before Exon A Indicator, between Exon A Indicator and Exon B Indicator, or after Exon B Indicator. For example:

| | |
|---|---|
| (1b) | Exon-exon junction indicator polynucleotide |
| | control: |

| | |
|---|---|
| AGGTCGGAGTCAACGG GCGGGTTATGCTGG | (SEQ ID NO:18) |
| TTTA | |
| Exon A Indicator | (SEQ ID NO:9) |
| Exon B Indicator (reverse) | (SEQ ID NO:19) |

| | |
|---|---|
| (1c) | Exon-exon junction indicator polynucleotide |
| | control: |

| | |
|---|---|
| AGGTCGGAGTCAACGG TAAACCAGCATAAC | (SEQ ID NO:20) |
| CCGC | |
| Exon A Indicator | (SEQ ID NO:9) |
| Exon B Indicator (complement) | (SEQ ID NO:21) |

| | |
|---|---|
| (1d) | Exon-exon junction indicator polynucleotide |
| | control: |

| | |
|---|---|
| AGGTCGGAGTCAACGG CGCCCAATACGACC | (SEQ ID NO:22) |
| AAAT | |
| Exon A Indicator | (SEQ ID NO:9) |
| Exon B Indicator (reverse | (SEQ ID NO:23) |
| complement) | |

In examples (1b), (1c) and (1d), indicator polynucleotide controls (SEQ ID NOs: 18, 20, 22) are shown that contain the reverse (SEQ ID NO:19), the complement (SEQ ID NO:21), and the reverse complement (SEQ ID NO:23), respectively, of Exon B Indicator (SEQ ID NO:14).

In one embodiment the IPC method provides an indicator polynucleotide control that contains the reverse, the complement, or the reverse complement of Exon A Indicator. In another embodiment the IPC method provides an indicator polynucleotide control that contains the reverse, the complement or the reverse complement of Exon B Indicator. In one embodiment the IPC method provides an indicator polynucleotide control that contains the reverse, the complement or the reverse complement of both Exon A Indicator and Exon B Indicator. In one embodiment, the IPC method provides an indicator polynucleotide control that contains more than two polynucleotides shuffled, reversed, complemented, or reverse-complemented.

In another embodiment, the IPC method provides an indicator polynucleotide control with at least one substituted nucleotide on each side of an exon-exon junction in an exon-exon junction indicator polynucleotide. For example:

(1e) Exon-exon junction indicator polynucleotide
control

| | |
|---|---|
| AGGTCCGAGTCAACGG ATTTGGTCGAATTGGGCG | (SEQ ID NO:24) |
| Exon A Indicator | (SEQ ID NO:25) |
| Exon B Indicator | (SEQ ID NO:26) |

The bases denoted in bold, underline represent nucleotide substitutions (‘mutations’, ‘point mutations’, ‘substitutions’, ‘base substitutions’, etc) relative to the indicator polynucleotide. The indicator polynucleotide control is comprised of a first portion (e.g. exon A indicator) that indicates a first exon and a second portion (e.g., exon B indicator) that indicates a second exon, where the first and second portions contain one or more nucleotide substitutions, relative to the indicator polynucleotide. The indicator polynucleotide control in this embodiment is also referred to as a ‘mutation control’. In one embodiment, the indicator polynucleotide control has an equal number of nucleotide substitutions on each side of the exon-exon junction; e.g., one nucleotide in each side of the first and second portions, or two nucleotides in each of the first and second portions. This indicator polynucleotide control is referred to herein as a ‘balanced mutation control’. In one embodiment, the substituted nucleotides in the mutation control or the balanced mutation control polynucleotides are complementary substitutions; i.e., G for C, C for G, A for T, T for A. In one embodiment, the indicator polynucleotide control comprises an additionally inserted nucleotide or comprises an omitted nucleotide from each of the first and second portions.

A device designed and/or constructed using the IPC method may be comprised of one or more polynucleotide pairs comprising an indicator polynucleotide and an indicator polynucleotide control as described in (1 a), (1b), (1c), (1d), (1e), or any combination thereof. In one embodiment, a device comprises one or more such pairs on a nucleotide array. In another embodiment, a device comprises one or more such pairs in containers or wells for multiplexed PCR.

Exon-Intron Junction Indicator Polynucleotide Controls

In the case of an exon-intron junction indicator polynucleotide, the IPC method, in one embodiment, provides an indicator polynucleotide comprised of polynucleotides indicating the exon and the intron in the exon-intron junction. For example:

| | |
|---|---|
| (2) | Exon-intron junction indicator |
| | polynucleotide: |

| | |
|---|---|
| AGGTCGGAGTCAACGG GTAACATTGACTGT | (SEQ ID NO:8) |
| CAA | |
| Exon Indicator | (SEQ ID NO:9) |
| Intron Indicator | (SEQ ID NO:10) |

| | |
|---|---|
| (2a) | Exon-intron junction indicator polynucleotide |
| | control: |

| | |
|---|---|
| GTAACATTGACTGTCAA AGGTCGGAGTCAA | (SEQ ID NO:27) |
| CGG | |
| Intron Indicator | (SEQ ID NO:10) |
| Exon Indicator | (SEQ ID NO:9) |

In the exon-intron junction indicator polynucleotide control (SEQ ID NO:27), the polynucleotide indicating the exon, Exon Indicator (SEQ ID NO:9), has been, in effect, moved to after the polynucleotide indicating the intron, Intron Indicator (SEQ ID NO:10), relative to the exon-intron junction indicator polynucleotide (SEQ ID NO:8). Exon Indicator (SEQ ID NO:9) has a 5′ end and a 3′ end, and Intron Indicator (SEQ ID NO:10) has a 5′ end and a 3′ end. The 5′ end of Exon Indicator (SEQ ID NO:9) is adjacent the 3′ end of Intron Indicator (SEQ ID NO:10) in the indicator polynucleotide control (SEQ ID NO:27). In contrast, in the exon-exon junction indicator polynucleotide (SEQ ID NO:8), the 5′ end of the Intron Indicator (SEQ ID NO:10) is adjacent the 3′ end of the Exon Indicator (SEQ ID NO:9). In essence, the position of the portions of the indicator polynucleotide control that identify the exons of the indicator polynucleotide are exchanged or “swapped”.

The exon-intron junction indicator polynucleotide control, as embodied by SEQ ID NO:27, is also referred to herein as a ‘swap control’ for the exon-intron junction indicator polynucleotide SEQ ID NO:8, in the sense that the position of the two portions of the exon-intron junction indicator polynucleotide are interchanged or “swapped”. More specifically, the exon-intron junction indicator polynucleotide comprises a first portion that indicates the 3′ end of an exon and a second portion that indicates the 5′ end of an intron region. The first portion precedes the second portion. The swap control comprises these same two portions, but ‘swapped’, so that the second portion comes before the first portion.

The relative lengths of Exon Indicator and Intron Indicator may be unequal, with length of the Exon Indicator less than or greater than the length of the Intron Indicator. It will also be appreciated that the length of the Exon Indicator can be equal to the length of the Intron Indicator.

The exon-intron junction indicator polynucleotide may contain one or more additional polynucleotides that do not indicate either the exon or the intron in the exon-intron junction. For example, the indicator polynucleotide may be formed by pre-pending a polynucleotide P_{A} before Exon Indicator, inserting a polynucleotide P_{AB} between Exon Indicator and Intron Indicator, or appending a polynucleotide P_{B} after Intron Indicator. In one embodiment, the IPC method creates an exon-intron junction indicator polynucleotide control that shuffles the order of the constituent polynucleotides, P_{A}, Exon Indicator, P_{AB}, Intron Indicator, and P_{B}. In one embodiment, the indicator polynucleotide control includes all of the constituent polynucleotides. In another embodiment, the indicator polynucleotide control includes an Exon Indicator and an Intron Indicator but does not include all of the other constituent polynucleotides.

In one embodiment the IPC method designs indicator polynucleotide controls that include reverse polynucleotides, reverse complement polynucleotides, or complement polynucleotides for Exon Indicator, Intron Indicator, or other polynucleotides before Exon Indicator, between Exon Indicator and Intron Indicator, or after Intron Indicator. For example:

| | |
|---|---|
| (2b) | Exon-intron junction indicator polynucleotide |
| | control: |

| | |
|---|---|
| AGGTCGGAGTCAACGG AACTGTCAGTTACA | (SEQ ID NO:28) |
| ATG | |
| Exon Indicator | (SEQ ID NO:9) |
| Intron Indicator (reverse) | (SEQ ID NO:29) |

| | |
|---|---|
| (2c) | Exon-intron junction indicator polynucleotide |
| | control: |

| | |
|---|---|
| AGGTCGGAGTCAACGG CATTGTAACTGACA | (SEQ ID NO:30) |
| GTT | |
| Exon Indicator | (SEQ ID NO:9) |
| Intron Indicator (complement) | (SEQ ID NO:31) |

| | |
|---|---|
| (2d) | Exon-intron junction indicator polynucleotide |
| | control: |

| | |
|---|---|
| AGGTCGGAGTCAACGG TTGACAGTCAATGT | (SEQ ID NO:32) |
| TAC | |
| Exon Indicator | (SEQ ID NO:9) |
| Intron Indicator (reverse | (SEQ ID NO:33) |
| complement) | |

In examples (2b), (2c) and (2d), indicator polynucleotide controls (SEQ ID NOs: 28, 30, 32) are shown that contain the reverse (SEQ ID NO:29), the complement (SEQ ID NO:31), and the reverse complement (SEQ ID NO:33), respectively, of Intron Indicator portion of the indicator polynucleotide control.

Accordingly, in one embodiment the IPC method provides indicator polynucleotide controls that contain the reverse, the complement or the reverse complement of Exon Indicator. In one embodiment, the IPC method provides indicator polynucleotide controls that contain the reverse, the complement, or the reverse complement of Intron Indicator. In another embodiment the IPC method provides indicator polynucleotide controls that contain the reverse, the complement or the reverse complement of both Exon Indicator and Intron Indicator. In yet another embodiment, the IPC method provides an indicator polynucleotide control that contains more than two polynucleotides shuffled, reversed, complemented, or reverse-complemented.

In one embodiment, the IPC method provides an indicator polynucleotide control with at least one substituted nucleotide on each side of an exon-intron junction. For example:

| | |
|---|---|
| (2e) | Exon-intron junction indicator polynucleotide |
| | control: |

| | |
|---|---|
| AGGTCCGAGTCAACGG GTAACATTGACAGTCAA | (SEQ ID NO:34) |
| Exon Indicator | (SEQ ID NO:25) |
| Intron Indicator | (SEQ ID NO:35) |

The characters highlighted in bold, underline represent nucleotide substitutions (‘mutations’, ‘point mutations’, ‘substitutions’, ‘base substitutions’, etc) relative to the indicator polynucleotide identified by SEQ ID NO:8. An indicator polynucleotide control containing such a nucleotide substitute is referred to herein as a ‘mutation control’. In one embodiment, the IPC method substitutes an equal number of nucleotides on each side of the junction, e.g., one nucleotide on each side or two nucleotides on each side. An indicator polynucleotide control containing an equal number of nucleotide substitutions in the exon indicator and the intron indicator is referred to herein as ‘balanced mutation control’. In one embodiment, the IPC method provides nucleotides substitutions that are complementary to the substituted nucleotides; i.e., G for C, C for G, A for T, T for A. In one embodiment, the IPC method inserts a nucleotide or removes a nucleotide from each side of the junction.

In one embodiment the IPC method combines the methods in (2a), (2b), (2c), (2d) and (2e).

A device designed and/or fabricated using indicator polynucleotide controls selected using the IPC method may consist of one or more polynucleotide pairs consisting of an indicator polynucleotide and an indicator polynucleotide control as described in (2a), (2b), (2c), (2d), (2e), or a combination thereof. In one embodiment, the IPC device may comprise one or more such pairs on a nucleotide array. In another embodiment, the IPC device may comprise one or more such pairs in containers or wells for multiplexed PCR.

Intron-Exon Junction Indicator Polynucleotide Controls

In the case of an intron-exon junction indicator polynucleotide, the IPC method, in one embodiment, selects an indicator polynucleotide containing the polynucleotides indicating the intron and exon in the intron-exon junction. For example:

| | |
|---|---|
| (3) | Intron-exon junction indicator |
| | polynucleotide: |

| | |
|---|---|
| ACATACGTTCTATTTAG ATTTGGTCGTATT | (SEQ ID NO:12) |
| GGGCG | |
| Intron Indicator | (SEQ ID NO:13) |
| Exon Indicator | (SEQ ID NO:14) |

| | |
|---|---|
| (3a) | Intron-exon junction indicator polynucleotide |
| | control: |

| | |
|---|---|
| ATTTGGTCGTATTGGGCG ACATACGTTCTA | (SEQ ID NO:36) |
| TTTAG | |
| Exon Indicator | (SEQ ID NO:14) |
| Intron Indicator | (SEQ ID NO:13) |

In the intron-exon junction indicator polynucleotide control (SEQ ID NO:36), the polynucleotide indicating the intron, Intron Indicator (SEQ ID NO:13), has been, in effect, moved to after the polynucleotide indicating the exon, Exon Indicator (SEQ ID NO:14),relative to the original intron-exon junction indicator polynucleotide (SEQ ID NO: 12).

An indicator polynucleotide control as exemplified by SEQ ID NO:36 is referred to herein as a ‘swap control’ for an intron-exon junction indicator polynucleotide, in the sense that in the control, the position of the two portions of the intron-exon junction indicator polynucleotide are interchanged or swapped, with respect to the position of these portions in the indicator polynucleotide (SEQ ID NO:12). That is, the intron-exon junction indicator polynucleotide comprises a first portion that indicates the 3′ end of an intron region and a second portion that indicates the 5′ end of an exon. The first portion precedes the second portion. The swap control comprises these same two portions, but ‘swapped’, so that the second portion comes before the first portion.

The relative lengths of Intron Indicator and Exon Indicator may be unequal, with the length of the Intron Indicator greater than or less then the length of the Exon Indicator. It will also be appreciated that the length of the Intron Indicator can be equal to the length of the Exon Indicator.

The intron-exon junction indicator polynucleotide may contain one or more additional polynucleotides that do not indicate either the exon or the intron in the exon-intron junction. For example, the indicator polynucleotide may be formed by pre-pending a polynucleotide PA before Intron Indicator, inserting a polynucleotide PAB between Intron Indicator and Exon Indicator, or appending a polynucleotide PB after Exon Indicator. In one embodiment, the IPC method creates an intron-exon junction indicator polynucleotide control that shuffles the order of the constituent polynucleotides, PA, Intron Indicator, PAB, Exon Indicator, and PB. In one embodiment, the indicator polynucleotide control includes all of the constituent polynucleotides. In another embodiment, the indicator polynucleotide control includes Intron Indicator and Exon Indicator but does not include all of the other constituent polynucleotides.

In one embodiment the IPC method designs indicator polynucleotide controls that include reverse polynucleotides, reverse complement polynucleotides, or complement polynucleotides for Intron Indicator, Exon Indicator or other polynucleotides before Intron Indicator, between Intron Indicator and Exon Indicator, or after Exon Indicator:

| | |
|---|---|
| (3b) | Intron-exon junction indicator polynucleotide |
| | control: |

| | |
|---|---|
| ACATACGTTCTATTTAG GCGGGTTATGCTG | (SEQ ID NO:37) |
| GTTTA | |
| Intron Indicator | (SEQ ID NO:13) |
| Exon Indicator (reverse) | (SEQ ID NO:19) |

| | |
|---|---|
| (3c) | Intron-exon junction indicator polynucleotide |
| | control: |

| | |
|---|---|
| ACATACGTTCTATTTAG CGCCCAATACGAC | (SEQ ID NO:38) |
| CAAAT | |
| Intron Indicator | (SEQ ID NO:13) |
| Exon Indicator (complement) | (SEQ ID NO:23) |

| | |
|---|---|
| (3d) | Intron-exon junction indicator polynucleotide |
| | control: |

| | |
|---|---|
| ACATACGTTCTATTTAG TAAACCAGCATAA | (SEQ ID NO:39) |
| CCCGC | |
| Intron Indicator | (SEQ ID NO:13) |
| Exon Indicator (reverse | (SEQ ID NO:21) |
| complement) | |

In (3b), (3c) and (3d), indicator polynucleotide controls (SEQ ID NOs:37, 38, 39) are shown that contain the reverse (SEQ ID NO:19), the complement (SEQ ID NO:23), and the reverse complement (SEQ ID NO:21), respectively, of Exon Indicator (SEQ ID NO:14).

In one embodiment the IPC method provides indicator polynucleotide controls that contain the reverse, the complement or the reverse complement of Intron Indicator. In one embodiment the IPC method provides indicator polynucleotide controls that contain the reverse, the complement or the reverse complement of Exon Indicator. In one embodiment the IPC method provides indicator polynucleotide controls that contain the reverse, the complement or the reverse complement of both Exon Indicator and Intron Indicator. In one embodiment, the IPC method provides an indicator polynucleotide control that contains more than two polynucleotides shuffled, reversed, complemented, or reverse-complemented.

In one embodiment, the IPC method provides an indicator polynucleotide control with at least one substituted nucleotide on each side of an intron-exon junction. For example:

| | |
|---|---|
| (3) | Intron-exon junction indicator polynucleotide |
| | control: |

| | |
|---|---|
| ACATACGATCTATTTAG ATTTGGTCGTATT | (SEQ ID NO:40) |
| GGCCG | |
| Intron Indicator | (SEQ ID NO:41) |
| Exon Indicator | (SEQ ID NO:42) |

The characters highlighted in bold, underline represent nucleotide substitutions (‘mutations’, ‘point mutations’, ‘substitutions’, ‘base substitutions’, etc) relative to the indicator polynucleotide. For example, Intron Indicator SEQ ID NO:41 has a single nucleotide substitution relative to the indicator polynucleotide SEQ ID NO:13. An indicator polynucleotide control having one or more nucleotide substitutions relative to the indicator polynucleotide is referred to as a ‘mutation control’. In one embodiment, the IPC method substitutes an equal number of nucleotides on each side of the junction, e.g., one nucleotide on each side or two nucleotides on each side of the junction. An indicator polynucleotide control thus prepared is referred to as a ‘balanced mutation control’. In one embodiment, the IPC method chooses nucleotides substitutions that are complementary to the substituted nucleotides; i.e., G for C, C for G, A for T, T for A. In one embodiment, the IPC method inserts a nucleotide or removes a nucleotide from each side of the junction.

In one embodiment the IPC method combines the methods in (3a), (3b), (3c), (3d) and (3e).

A device comprising indicator polynucleotides and indicator polynucleotide controls selected using the IPC method may consist of one or more polynucleotide pairs consisting of an indicator polynucleotide and an indicator polynucleotide control as described in (3a), (3b), (3c), (3d), (3e), or a combination thereof. In one embodiment, the IPC device may comprise one or more such pairs on a nucleotide array. In another embodiment, the IPC device may comprise one or more such pairs in containers or wells for multiplexed PCR.

Exon or Intron Indicator Polynucleotide Controls

In another embodiment, the method is used for identification of an indicator polynucleotide control for an exon or intron indicator polynucleotide. The method selects an indicator polynucleotide containing two or more subsequences of the exon or intron indicator polynucleotide. For example:

| | |
|---|---|
| (4) | Exon or intron indicator polynucleotide: |

(SEQ ID NO:43)

CTGAAGTGGAGCAGGTAC AGTCACAGCTGTGGACAG

(SEQ ID NO:44)

Polynucleotide A

(SEQ ID NO:45)

Polynucleotide B

| | |
|---|---|
| (4a) | Exon or intron indicator polynucleotide: |
| | control: |

(SEQ ID NO:46)

AGTCACAGCTGTGGACAG CTGAAGTGGAGCAGGTAC

(SEQ ID NO:45)

Polynucleotide A

(SEQ ID NO:44)

Polynucleotide B

In the exon or intron indicator polynucleotide control (SEQ ID NO:46), Polynucleotide A (SEQ ID NO:44) has been, in effect, moved to after Polynucleotide B (SEQ ID NO:45) relative to the original exon or intron indicator polynucleotide (SEQ ID NO:43). The indicator polynucleotide control is a ‘swap control’ for the exon or intron indicator polynucleotide, in the sense that it swaps two portions of the exon or intron indicator polynucleotide. The exon or intron indicator polynucleotide comprises a first portion that indicates a first portion of an exon or intron and a second portion that indicates a second portion of the exon or intron. The first portion precedes the second portion. The swap control comprises these same two portions, but ‘swapped’, so that the second portion comes before the first portion.

The relative lengths of Polynucleotide A and Polynucleotide B may be unequal, with the length of Polynucleotide A less than the length of Polynucleotide B, or the length of Polynucleotide A greater than the length of Polynucleotide B. It will also be appreciated that the length of Polynucleotide A can be equal to the length of Polynucleotide B.

In one embodiment, the IPC method creates an exon or intron indicator polynucleotide control that shuffles the order of the constituent polynucleotides, Polynucleotide A, Polynucleotide B, and other polynucleotides Pᵢ. In one embodiment, the indicator polynucleotide control includes all of the constituent polynucleotides. In another embodiment, the indicator polynucleotide control includes only some of the constituent polynucleotides.

In one embodiment the IPC method chooses indicator polynucleotide controls that include reverse polynucleotides, reverse complement polynucleotides, or complement polynucleotides for constituent polynucleotides of the indicator polynucleotide. For example:

| | |
|---|---|
| (4b) | Exon or intron indicator polynucleotide |
| | control: |

(SEQ ID NO:47)

CTGAAGTGGAGCAGGTAC GACAGGTGTCGACACTGA

(SEQ ID NO:44)

Polynucleotide A

(SEQ ID NO:48)

Polynucleotide B (reverse)

| | |
|---|---|
| (4c) | Exon or intron indicator polynucleotide |
| | control: |

(SEQ ID NO:49)

CTGAAGTGGAGCAGGTAC TCAGTGTCGACACCTGTC

(SEQ ID NO:44)

Polynucleotide A

(SEQ ID NO:50)

Polynucleotide B (complement)

| | |
|---|---|
| (4d) | Exon or intron indicator polynucleotide |
| | control: |

(SEQ ID NO:51)

CTGAAGTGGAGCAGGTAC CTGTCCACAGCTGTGACT

(SEQ ID NO:44)

Polynucleotide A

(SEQ ID NO:52)

Polynucleotide B (reverse complement)

In (4b), (4c) and (4d), indicator polynucleotide controls (SEQ ID NOs:47, 49, 51) are shown that contain the reverse (SEQ ID NO:48), the complement (SEQ ID NO:50), and the reverse complement (SEQ ID NO:52), respectively, of Polynucleotide B (SEQ ID NO:45).

Accordingly, in one embodiment the IPC method provides an indicator polynucleotide control that contains the reverse, the complement or the reverse complement of Polynucleotide A. In one embodiment the IPC method provides an indicator polynucleotide control that contains the reverse, the complement or the reverse complement of Polynucleotide B. In one embodiment the IPC method provides an indicator polynucleotide control that contains the reverse, the complement or the reverse complement of both Polynucleotide A and Polynucleotide B. In one embodiment, the IPC method provides an indicator polynucleotide control that contains more than two polynucleotides shuffled, reversed, complemented, or reverse-complemented.

In one embodiment the IPC method combines the methods in (4a), (4b), (4c) and (4d).

In other embodiments, the IPC method may combine the methods above with other methods that occur to one skilled in the arts.

A device designed and/or constructed using the IPC method may comprise one or more polynucleotide pairs consisting of an indicator polynucleotide and an indicator polynucleotide control as described in (4a), (4b), (4c), (4d), or a combination thereof, on a nucleotide array. In another embodiment, the IPC device may comprise one or more such pairs in containers or wells for multiplexed PCR.

The examples above show indicator polynucleotides with the same strand as the indicated polynucleotides, e.g., both are sense strand or both are anti-sense strand. In performing an experiment on a nucleotide array, a sense strand indicator polynucleotide can be used on the array and a complementary RNA or complementary DNA can be created to hybridize to the indicator polynucleotide on the array. In this case, the indicated polynucleotide can be viewed to be a portion of the sense strand RNA, commonly referred to as a splice variant, a splice isoform, mRNA, RNA, and the like. However, it will be appreciated that this arrangement set forth in the examples above is shown for illustrative purposes and that the method, array, and techniques are not limited to this scenario. It is also possible to design indicator polynucleotides and indicator polynucleotide controls that are complementary to an expected or predicted indicator polynucleotide in a sample.

One skilled in the art will also appreciate that the indicator polynucleotide may be forward or reverse strand depending on the sample preparation and hybridization protocols. Likewise, the indicated polynucleotide may be forward or reverse strand depending on the sample preparation and hybridization protocols. Ultimately, a hybridization reaction occurs between a polynucleotide and a polynucleotide complementary, or approximately complementary, to it.

In the case of PCR, one skilled in the art will appreciate that a first indicator polynucleotide primer may be forward strand, and a second indicator polynucleotide primer may be reverse strand, or vice versa, again depending upon the experimental protocols employed. The indicated polynucleotides may be forward or reverse strand accordingly.

Storage Medium Comprising Data for Indicator Polynucleotide Controls

In another embodiment, a storage medium containing data obtained from a study using an indicator polynucleotide control, as described above, is provided. A storage medium containing data obtained from a study using one or more pairs comprising an indicator polynucleotide and an indicator polynucleotide control is also contemplated.

The storage medium, for example, can contain expression level data for one or more indicator polynucleotide controls, such as swap controls or balanced mutation controls. The storage medium may take the form of, for example, a computer hard drive, a memory device, a compact disc (CD), a digital video disk or digital versatile disk (DVD), computer cache, paper, magnetic tape, or any medium capable of temporary or permanent storage of a data set, data representation, or a computer file. The computer file can be, for example, a tab-delimited or csv file, a nucleotide sequence file, a spreadsheet file, a database table, an XML document, an HTML table, or the like. The computer file may be binary, ascii, encrypted, encoded, etc. The data representation may comprise a portion of a file (e.g., part of a database) or it may span multiple files (e.g., a normalized database comprising multiple data tables stored in separate locations). The data representation may comprise print material, such as a computer printout or lab notebook. The data may be used for data analysis purposes, for archival purposes, etc.

In one embodiment, the data contained on the storage medium may comprise an identifier for an indicator polynucleotide; an identifier for an indicator polynucleotide control; a numerical value, such as an expression level, for the indicator polynucleotide; a numerical value, such as an expression level, for the indicator polynucleotide control; a sequence for the indicator polynucleotide; and/or a sequence for the indicator polynucleotide control. The data may include other information such as a public database identifier; an exon index, intron index, pair of exon indexes or an exon index and an intron index; a background level from an array experiment, a mean or median value from a fluorescent scanner; a number of pixels; a flag for sequence quality or presence of homologues; or other information that may occur to one of ordinary skill in the art. The data set may comprise any combination of these types of information. The data set may comprise such information for a plurality of pairs. The representation may comprise such data for 100 or more pairs, or 1,000 or more pairs, or 10,000 or more pairs, or 100,000 or more pairs, or 1,000,000 or more pairs.

The data may be transmitted via a networking protocol such as UDP, TCP-IP, FTP or HTTP. Alternatively, the data may be transmitted via domestic mail, international mail, courier, etc.

Analysis Using Indicator Polynucleotide Controls

In one embodiment, the IPC method provides an indicator polynucleotide and one or more indicator polynucleotide controls to determine the amount of an indicated polynucleotide in a sample. In one embodiment, data from a study using a nucleotide array constructed and/or designed using the IPC method is analyzed using an algorithm of the form:
A=F(Aᵢ,A_{c}) Equation (1)
where A is an amount of the indicated polynucleotide, a signal to noise ratio, or another value of use in assessing the measure of the indicated polynucleotide; F is an equation; Aᵢ is the amount measured by the indicator polynucleotide (possibly after normalizing, subtracting background or otherwise modifying the raw value representing the measured amount); and A_{c} is the amount measured by the indicator polynucleotide controls (possibly after normalizing, subtracting background or otherwise modifying).

In one embodiment, an equation F that subtracts the amounts of Aᵢ and A_{c} is used:
F(Aᵢ,A_{c})=Aᵢ−A_{c} Equation (2)
In another embodiment, an equation F that subtracts the logs of Aᵢ and A_{c} is used
F(Aᵢ,A_{c})=log Aᵢ₋log A_{c} Equation (3)
In another embodiment, an equation F that takes a ratio of Aᵢ and A_{c} is used
F(Aᵢ,A_{c})=Aᵢ/A_{c} Equation (4)
In another embodiment, an equation F that takes a ratio of the log of Aᵢ and the log of A_{c} is used
F(Aᵢ,A_{c})=log Aᵢ/log A_{c} Equation (5)
In another embodiment, an equation F that computes a signal to noise ratio, S, for Aᵢ, is used, for example:
S=G(Aᵢ,A_{c})=(Aᵢ−A_{c})/Aᵢ Equation (6)
where G is an equation. In one embodiment, expression level values from one or more pairs of indicator polynucleotides and control indicator polynucleotides in conjunction with a cutoff value for filtering data are used. The indicator polynucleotides that fail to pass the cutoff (e.g., A<=cutoff) are excluded from analysis. Equivalently, indicator polynucleotides that do pass the cutoff are included in analysis. A filter, such as signal-to-noise ratio greater than one, greater than 1.5, greater than 2, greater than 2.5, etc, can be used to determine if a target polynucleotide is expressed in a sample.

In an embodiment, expression level values from one or more pairs are used, each pair comprising an indicator polynucleotide and an indicator polynucleotide control, such as a swap control, and assigns a weight W to each indicator polynucleotide.

An equation involving an expression level for an indicator polynucleotide control by means of a computer software program can also be used. The analysis method can be used in a device such as a scanner, for example, to read labeling applied to polynucleotides in a sample.

In other embodiments, other equations that occur to one skilled in the art can be used for analysis. For example, the analysis may use the expression level data resulting from an equation above as inputs to a system of linear equations or a deconvolution algorithm to determine amounts of splice variants in one or more samples. An exemplary algorithm and method is described in U.S. Publication Nos. 2003/0087261 and 2004/0076959, which are incorporated by reference herein. The analysis may combine the equation into the system of linear equations or deconvolution algorithm, by substituting the variables from the former equation into the latter system or algorithm.

The analysis may use the expression levels resulting from an equation to determine if a splice variant of a gene is expressed in a sample. For example, suppose expression data is obtained for one or more pairs of indicator polynucleotides, each pair comprising an indicator polynucleotide for an exon, intron, exon-exon junction, exon-intron junction or intron-exon junction of a splice variant of a gene and an indicator polynucleotide control. The method computes one or more values such as signal-to-noise ratios, log ratios, ratios or differences, for one or more of the pairs. In an embodiment, such value or values are used to determine if a splice variant of a gene is expressed. In another embodiment, the value or values are used to determine an expression level for the splice variant of the gene. It will be appreciated that the analysis can be performed for one or more splice variants of one or more genes.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A nucleotide array, comprising: (i) an indicator polynucleotide for a target polynucleotide in a sample, the indicator polynucleotide selected from the group consisting of an indicator polynucleotide for an exon-exon junction, an indicator polynucleotide for an exon-intron junction, an indicator polynucleotide for an intron-exon junction, indicator polynucleotide for an exon and an indicator polynucleotide for an intron, and (ii) an indicator polynucleotide control for the indicator polynucleotide, the indicator polynucleotide control selected from the group consisting of a swap control and a balanced mutation control.

2. A nucleotide array comprising a plurality of pairs of indicator polynucleotides, each pair comprising: (i) an indicator polynucleotide for a target polynucleotide in a sample, the indicator polynucleotide selected from the group consisting of an indicator polynucleotide for an exon-exon junction, an indicator polynucleotide for an exon-intron junction, an indicator polynucleotide for an intron-exon junction, indicator polynucleotide for an exon and an indicator polynucleotide for an intron, and (ii) an indicator polynucleotide control for the indicator polynucleotide, the indicator polynucleotide control selected from the group consisting of a swap control and a balanced mutation control.

3. The nucleotide array of claim 1, comprising at least about 100 of said pairs.

4. A storage medium comprising data obtained from a study using a nucleotide array according to claim 1.

5. The storage medium of claim 4, wherein the data is stored in a portion of a computer file.

6. The storage medium of claim 4, wherein the data is stored in one or more computer files.

7. A storage medium comprising data obtained from a study using a nucleotide array according to claim 2.

8. The storage medium of claim 7, wherein the data is stored in a portion of a computer file.

9. The storage medium of claim 7, wherein the data is stored in one or more computer files.

10. A method of determining an expression level for a target exon-exon junction, exon-intron junction, or intron-exon junction polynucleotide in a sample, comprising:
obtaining expression level data for a sample for (i) an indicator polynucleotide for a target polynucleotide in the sample, the indicator polynucleotide selected from the group consisting of an indicator polynucleotide for an exon-exon junction, an indicator polynucleotide for an exon-intron junction, an indicator polynucleotide for an intron-exon junction, an indicator polynucleotide for an exon, and an indicator polynucleotide for an intron, and (ii) an indicator polynucleotide control for the indicator polynucleotide, the indicator polynucleotide control selected from the group consisting of a swap control and a mutation control, and
applying a mathematical algorithm to the expression level data to determine an expression level for the target polynucleotide in the sample.

11. A method of determining if a splice variant of a gene is expressed in a sample, comprising:
obtaining expression level data for a sample for (i) an indicator polynucleotide for a target polynucleotide in the sample, the indicator polynucleotide selected from the group consisting of an indicator polynucleotide for an exon-exon junction, an indicator polynucleotide for an exon-intron junction, an indicator polynucleotide for an intron-exon junction, an indicator polynucleotide for an exon, and an indicator polynucleotide for an intron, and (ii) an indicator polynucleotide control for the indicator polynucleotide, the indicator polynucleotide control selected from the group consisting of a swap control and a mutation control, and
applying a mathematical algorithm to the expression level data to determine if the splice variant of the gene is expressed in the sample.

12. A method of determining an expression level for one or more expected splice variants of a gene in a sample, comprising:
obtaining expression level data for a sample for (i) an indicator polynucleotide for a target polynucleotide in the sample, the indicator polynucleotide selected from the group consisting of an indicator polynucleotide for an exon-exon junction, an indicator polynucleotide for an exon-intron junction, an indicator polynucleotide for an intron-exon junction, an indicator polynucleotide for an exon, and an indicator polynucleotide for an intron, and (ii) an indicator polynucleotide control for the indicator polynucleotide, the indicator polynucleotide control selected from the group consisting of a swap control and a mutation control, and
applying a mathematical algorithm to the expression level data to determine an expression level for each of the expected splice variants of the gene in the sample.


SEQUENCE LISTING (52 sequences)

SEQ ID NO: 1 - DNA, 40 bp, Artificial Sequence
  Synthetic Polynucleotide
agaagtatggcccgatttacagatggacttgccaccagtg

SEQ ID NO: 2 - DNA, 20 bp, Artificial Sequence
  Synthetic Polynucleotide
agaagtatggcccgatttac

SEQ ID NO: 3 - DNA, 20 bp, Artificial Sequence
  Synthetic Polynucleotide
agatggacttgccaccagtg

SEQ ID NO: 4 - DNA, 38 bp, Artificial Sequence
  Synthetic Polynucleotide
agaagtatggcccgatttacagggagaagctcggcaac

SEQ ID NO: 5 - DNA, 18 bp, Artificial Sequence
  Synthetic Polynucleotide
agggagaagctcggcaac

SEQ ID NO: 6 - DNA, 40 bp, Artificial Sequence
  Synthetic Polynucleotide
agtagcatagctcgatttacagatggacttgccaccagtg

SEQ ID NO: 7 - DNA, 20 bp, Artificial Sequence
  Synthetic Polynucleotide
agtagcatagctcgatttac

SEQ ID NO: 8 - DNA, 33 bp, Artificial Sequence
  Synthetic Polynucleotide
aggtcggagtcaacgggtaacattgactgtcaa

SEQ ID NO: 9 - DNA, 16 bp, Artificial Sequence
  Synthetic Polynucleotide
aggtcggagtcaacgg

SEQ ID NO: 10 - DNA, 17 bp, Artificial Sequence
  Synthetic Polynucleotide
gtaacattgactgtcaa

SEQ ID NO: 11 - DNA, 40 bp, Artificial Sequence; identical to SEQ ID NO: 1
  Synthetic Polynucleotide

SEQ ID NO: 12 - DNA, 35 bp, Artificial Sequence
  Synthetic Polynucleotide
acatacgttctatttagatttggtcgtattgggcg

SEQ ID NO: 13 - DNA, 17 bp, Artificial Sequence
  Synthetic Polynucleotide
acatacgttctatttag

SEQ ID NO: 14 - DNA, 18 bp, Artificial Sequence
  Synthetic Polynucleotide
atttggtcgtattgggcg

SEQ ID NO: 15 - DNA, 38 bp, Artificial Sequence
  Synthetic Polynucleotide
agaagtatggcccgatttacatttggtcgtattgggcg

SEQ ID NO: 16 - DNA, 34 bp, Artificial Sequence
  Synthetic Polynucleotide
aggtcggagtcaacggatttggtcgtattgggcg

SEQ ID NO: 17 - DNA, 34 bp, Artificial Sequence
  Synthetic Polynucleotide
atttggtcgtattgggcgaggtcggagtcaacgg

SEQ ID NO: 18 - DNA, 34 bp, Artificial Sequence
  Synthetic Polynucleotide
aggtcggagtcaacgggcgggttatgctggttta

SEQ ID NO: 19 - DNA, 18 bp, Artificial Sequence
  Synthetic Polynucleotide
gcgggttatgctggttta

SEQ ID NO: 20 - DNA, 34 bp, Artificial Sequence
  Synthetic Polynucleotide
aggtcggagtcaacggtaaaccagcataacccgc

SEQ ID NO: 21 - DNA, 18 bp, Artificial Sequence
  Synthetic Polynucleotide
taaaccagcataacccgc

SEQ ID NO: 22 - DNA, 34 bp, Artificial Sequence
  Synthetic Polynucleotide
aggtcggagtcaacggcgcccaatacgaccaaat

SEQ ID NO: 23 - DNA, 18 bp, Artificial Sequence
  Synthetic Polynucleotide
cgcccaatacgaccaaat

SEQ ID NO: 24 - DNA, 34 bp, Artificial Sequence
  Synthetic Polynucleotide
aggtccgagtcaacggatttggtcgaattgggcg

SEQ ID NO: 25 - DNA, 16 bp, Artificial Sequence
  Synthetic Polynucleotide
aggtccgagtcaacgg

SEQ ID NO: 26 - DNA, 18 bp, Artificial Sequence
  Synthetic Polynucleotide
atttggtcgaattgggcg

SEQ ID NO: 27 - DNA, 33 bp, Artificial Sequence; identical to SEQ ID NO: 8
  Synthetic Polynucleotide

SEQ ID NO: 28 - DNA, 33 bp, Artificial Sequence
  Synthetic Polynucleotide
gtaacattgactgtcaaaggtcggagtcaacgg

SEQ ID NO: 29 - DNA, 17 bp, Artificial Sequence
  Synthetic Polynucleotide
aactgtcagttacaatg

SEQ ID NO: 30 - DNA, 33 bp, Artificial Sequence
  Synthetic Polynucleotide
aggtcggagtcaacggcattgtaactgacagtt

SEQ ID NO: 31 - DNA, 17 bp, Artificial Sequence
  Synthetic Polynucleotide
cattgtaactgacagtt

SEQ ID NO: 32 - DNA, 33 bp, Artificial Sequence
  Synthetic Polynucleotide
aggtcggagtcaacggttgacagtcaatgttac

SEQ ID NO: 33 - DNA, 17 bp, Artificial Sequence
  Synthetic Polynucleotide
ttgacagtcaatgttac

SEQ ID NO: 34 - DNA, 33 bp, Artificial Sequence
  Synthetic Polynucleotide
aggtccgagtcaacgggtaacattgacagtcaa

SEQ ID NO: 35 - DNA, 17 bp, Artificial Sequence
  Synthetic Polynucleotide
gtaacattgacagtcaa

SEQ ID NO: 36 - DNA, 35 bp, Artificial Sequence
  Synthetic Polynucleotide
atttggtcgtattgggcgacatacgttctatttag

SEQ ID NO: 37 - DNA, 35 bp, Artificial Sequence
  Synthetic Polynucleotide
acatacgttctatttaggcgggttatgctggttta

SEQ ID NO: 38 - DNA, 35 bp, Artificial Sequence
  Synthetic Polynucleotide
acatacgttctatttagcgcccaatacgaccaaat

SEQ ID NO: 39 - DNA, 35 bp, Artificial Sequence
  Synthetic Polynucleotide
acatacgttctatttagtaaaccagcataacccgc

SEQ ID NO: 40 - DNA, 35 bp, Artificial Sequence
  Synthetic Polynucleotide
acatacgatctatttagatttggtcgtattggccg

SEQ ID NO: 41 - DNA, 17 bp, Artificial Sequence
  Synthetic Polynucleotide
acatacgatctatttag

SEQ ID NO: 42 - DNA, 18 bp, Artificial Sequence
  Synthetic Polynucleotide
atttggtcgtattggccg

SEQ ID NO: 43 - DNA, 36 bp, Artificial Sequence
  Synthetic Polynucleotide
ctgaagtggagcaggtacagtcacagctgtggacag

SEQ ID NO: 44 - DNA, 18 bp, Artificial Sequence
  Synthetic Polynucleotide
ctgaagtggagcaggtac

SEQ ID NO: 45 - DNA, 18 bp, Artificial Sequence
  Synthetic Polynucleotide
agtcacagctgtggacag

SEQ ID NO: 46 - DNA, 36 bp, Artificial Sequence
  Synthetic Polynucleotide
agtcacagctgtggacagctgaagtggagcaggtac

SEQ ID NO: 47 - DNA, 36 bp, Artificial Sequence
  Synthetic Polynucleotide
ctgaagtggagcaggtacgacaggtgtcgacactga

SEQ ID NO: 48 - DNA, 18 bp, Artificial Sequence
  Synthetic Polynucleotide
gacaggtgtcgacactga

SEQ ID NO: 49 - DNA, 36 bp, Artificial Sequence
  Synthetic Polynucleotide
ctgaagtggagcaggtactcagtgtcgacacctgtc

SEQ ID NO: 50 - DNA, 18 bp, Artificial Sequence
  Synthetic Polynucleotide
tcagtgtcgacacctgtc

SEQ ID NO: 51 - DNA, 36 bp, Artificial Sequence
  Synthetic Polynucleotide
ctgaagtggagcaggtacctgtccacagctgtgact

SEQ ID NO: 52 - DNA, 18 bp, Artificial Sequence
  Synthetic Polynucleotide
ctgtccacagctgtgact